(19) United States
(12) Patent Application Publication
(10) Pub. No.: US 2026/0022425 A1
(43) Pub. Date: Jan. 22, 2026

(54) KIT OF DETECTING MYCOPLASMA PNEUMONIAE AND METHOD THEREFOR

(71) Applicant: Delta Electronics, Inc., Taoyuan City (TW)

(72) Inventors: Meng Jun FU, Taoyuan City (TW); Hsiu Hui CHANG, Taoyuan City (TW); Yu Han SHEN, Taoyuan City (TW)

(21) Appl. No.: 18/894,038

(22) Filed: Sep. 24, 2024

(30) Foreign Application Priority Data
Jul. 18, 2024 (CN) 202410964985.3

(51) Int. Cl. C12Q 1/689

(52) U.S. Cl. CPC C12Q 1/689

(57) ABSTRACT

A kit of detecting Mycoplasma pneumoniae is provided, and the kit comprises a combination of primers and probe consisting of a forward primer, a reverse primer and a probe. The forward primer is selected from the group consisting of SEQ ID NO: 2-3 and complementary sequences thereof, the reverse primer is selected from the group consisting of SEQ ID NO: 6-8 and complementary sequences thereof, and the probe is SEQ ID NO: 9 or a complementary sequence thereof. A method for detecting Mycoplasma pneumoniae using the above kit is also provided.

The official copy of sequence listing is submitted concurrently with the specification as an XML file with a file name of TP240334-US SEQ.xml, a creation date of Aug. 7, 2024 and a size of 11 KB. This sequence listing is part of the specification and is hereby incorporated in its entirely by reference herein.

### FIELD OF INVENTION

The present disclosure relates to a kit for detecting Mycoplasma pneumoniae and a method therefor.

### BACKGROUND OF INVENTION

Mycoplasma pneumoniae is a type of Mycoplasma and is one of the common pathogens causing respiratory tract infections. Symptoms of common infections are mild, with symptoms similar to those of a common cold, including sore throat, headache, fever, and a cough that lasts for weeks to months. If children under five years old or adults with low immunity are infected, there are extra symptoms, such as nasal congestion, runny nose, shortness of breath, vomiting, and diarrhea. When humans have a mild infection, they will recover on their own. However, when humans have a severe infection, the severe infection will turn into atypical pneumonia, with additional symptoms, such as chills, mucus sputum, chest pain, and difficulty breathing. In addition, it may cause other serious complications, such as severe pneumonia, encephalitis, hemolytic anemia, renal insufficiency, skin lesions.

Since Mycoplasma pneumoniae does not have a cell wall, common antibiotics that inhibit cell wall synthesis, such as beta-lactam antibiotics, are ineffective against Mycoplasma pneumoniae. Therefore, the best choice for treatment is macrolide antibiotics and alternative drugs, such as quinolone or tetracycline antibiotics. In order to avoid the increase of drug-resistant bacteria caused by the misuse of antibiotics, it is necessary to detect and confirm the pathogen causing the disease before using drugs accurately.

Current methods for detecting Mycoplasma pneumoniae including bacterial culture, cold agglutinin, serologic diagnosis and nucleic acid detection, the sensitivity and specificity of nucleic acid detection are higher, and the results can be obtained quickly, which is convenient for doctors to diagnose. Although the nucleic acid detection kits on the market have a certain degree of specificity, the probability of false positives or false negatives is still high, and most of the nucleic acid detection kits on the market still require about an hour or even longer to perform nucleic acid amplification and detection, affecting the accuracy of medication and delaying the timing of treatment.

In the light of the above, it is necessary to provide a nucleic acid detection kit and method that is different from those of prior art to solve the problems of conventional technologies.

### SUMMARY OF INVENTION

Therefore, the primary object of the present disclosure is to provide a kit of detecting Mycoplasma pneumoniae, comprising a combination of primers and probe consisting of a forward primer, a reverse primer and a probe, wherein the forward primer is selected from the group consisting of SEQ ID NO: 2-3 and complementary sequences thereof, the reverse primer is selected from the group consisting of SEQ ID NO: 6-8 and complementary sequences thereof, and the probe is SEQ ID NO: 9 or a complementary sequence thereof.

In some embodiments, the forward primer is SEQ ID NO: 2, and the reverse primer is SEQ ID NO: 8.

In some embodiments, the 5′ end of the probe is connected to a reporter, and the 3′ end of the probe is connected to a quencher.

In some embodiments, the reporter is Cy5.

In some embodiments, the combination of primers and probe is in a form of a freeze-dried reagent.

Another object of the present disclosure is to provide a method for detecting Mycoplasma pneumoniae, comprising steps of:
S110: providing a biological sample;
S120: providing the kit of detecting Mycoplasma pneumoniae as claimed in any one of claims 1 to 5;
S130: mixing the biological sample and the kit of detecting Mycoplasma pneumoniae to obtain a reaction solution;
S140: amplifying nucleic acids in the biological sample by performing a polymerase chain reaction using the reaction solution to obtain an amplification product; and
S150: detecting a fluorescent signal after each amplification cycle, analyzing the amplification product based on the fluorescent signal to determine whether Mycoplasma pneumoniae is present in the biological sample, wherein when the fluorescent signal is detected, it is determined that Mycoplasma pneumoniae is present in the biological sample; when the fluorescent signal is not detected, it is not yet determined that Mycoplasma pneumoniae is present in the biological sample, and performing a next amplification cycle.

In some embodiments, the polymerase chain reaction is real-time quantitative polymerase chain reaction (qPCR).

In some embodiments, the polymerase chain reaction includes 40 to 50 of the amplification cycles.

In some embodiments, each of the amplification cycles comprises:
a denaturation reaction, performed at 93 to 97° C. for 4 to 6 seconds; and an annealing and extension reaction, performed at 58 to 62° C. for 8 to 12 seconds.

In some embodiments, an enzyme activation reaction is performed prior to each of the amplification cycles, and the enzyme activation reaction is performed at 93 to 97° C. for 25 to 35 seconds.

Compared with prior art, the advantages of the present disclosure are that:
(1) High specificity: the sequence coverage rate of primers and probes is maintained at more than 99%, gene sequence fragments of other species other than Mycoplasma pneumoniae is not identified, and thus the kit has a high degree of specificity;
(2) High sensitivity: the limit of detection (LoD) of this primer-probe combination is 5 copy/rxn nucleic acid samples; and when there is interference from human genome DNA, this primer-probe combination can still detect 100 copy/rxn nucleic acid samples;
(3) Fast reaction time: compared with other detection kits in the industry, the detection kit of the present disclosure has a reaction time of less than 1 hour, and the results can be obtained quickly; and
(4) Cost reduction: after the primer-probe combination is freeze-dried, the reaction efficiency is not be affected, and the cold chain cost during transportation can be reduced.

### DESCRIPTION OF DRAWINGS

For a more complete understanding of the embodiments and their advantages, reference is now made to the following description taken in conjunction with the accompanying drawings, in which:

FIG. 1 is a flow chart of the method for detecting Mycoplasma pneumoniae according to the present disclosure;

FIG. 2 shows the partial sequence of Mycoplasma pneumoniae P1 gene and the design positions of primers and probes;

FIGS. 3A and 3B are respectively an amplification curve graph and a standard curve graph obtained after the qPCR reaction using the kit of detecting Mycoplasma pneumoniae according to the present disclosure; and

FIG. 4 is a picture of the gel electrophoresis of the product after the qPCR reaction using the kit of detecting Mycoplasma pneumoniae according to the present disclosure.

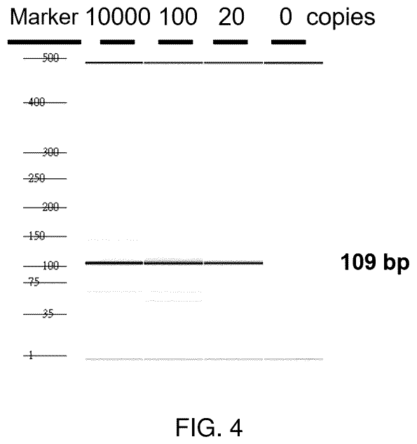

### DETAILED DESCRIPTION OF PREFERRED EMBODIMENTS

Some specific embodiments according to the present disclosure will be described below; however, without departing from the spirit of the present disclosure, the present disclosure can still be practiced in various forms, and the protection scope of the present disclosure should not be construed as being limited to what is stated in the specification. In addition, unless otherwise indicated in the context, “a”, “the” and similar terms used in the specification (especially in the appended claims) should be understood to include both the singular and the plural forms.

Although a series of operations or steps are used below to illustrate the method disclosed herein, the order shown in these operations or steps should not be construed as a limitation of the present disclosure. For example, certain operations or steps may be performed in a different order and/or concurrently with other steps. Furthermore, not all operations, steps, and/or features must be performed to practice embodiments of the present disclosure. Additionally, each operation or step described herein may include several sub-steps or actions.

The embodiment of the present disclosure provide a kit of detecting Mycoplasma pneumoniae, comprising a combination of primers and probe consisting of a forward primer, a reverse primer and a probe, wherein the forward primer is selected from the group consisting of SEQ ID NO: 2-3 and complementary sequences thereof, the reverse primer is selected from the group consisting of SEQ ID NO: 6-8 and complementary sequences thereof, and the probe is SEQ ID NO: 9 or a complementary sequence thereof.

In a specific embodiment, the present disclosure uses SEQ ID NO: 2 as the forward primer, and SEQ ID NO: 8 as the reverse primer, and SEQ ID NO: 9 as the probe to serve as the combination of primers and probe in the kit of detecting Mycoplasma pneumoniae of the present disclosure.

In a specific embodiment, the 5′ end of the probe is connected to a reporter, and the 3′ end of the probe is connected to a quencher. The types of the reporter and the quencher are not limited, as long as they can stably emit fluorescent signals and do not affect the function of the probe. For example, the reporter may be Cy5.

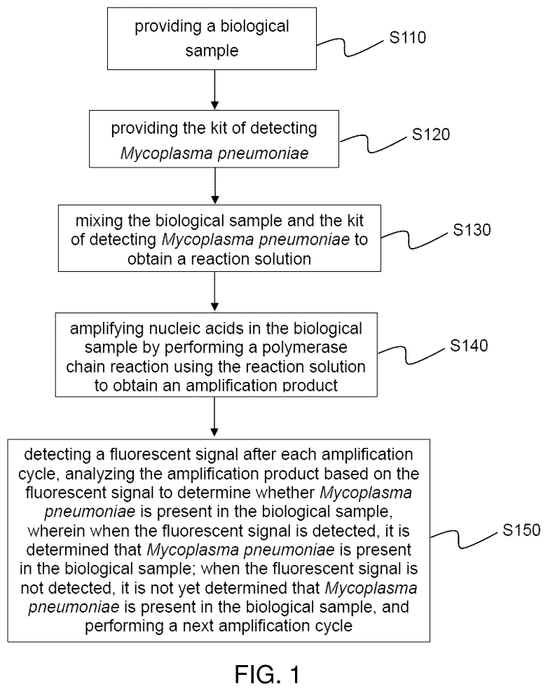

Another embodiment of the present disclosure provides a method for detecting Mycoplasma pneumoniae. As shown in FIG. 1, the method comprises steps of:
S110: providing a biological sample;
S120: providing the kit of detecting Mycoplasma pneumoniae as claimed in any one of claims 1 to 5;
S130: mixing the biological sample and the kit of detecting Mycoplasma pneumoniae to obtain a reaction solution;
S140: amplifying nucleic acids in the biological sample by performing a polymerase chain reaction using the reaction solution to obtain an amplification product; and
S150: detecting a fluorescent signal after each amplification cycle, analyzing the amplification product based on the fluorescent signal to determine whether Mycoplasma pneumoniae is present in the biological sample, wherein when the fluorescent signal is detected, it is determined that Mycoplasma pneumoniae is present in the biological sample; when the fluorescent signal is not detected, it is not yet determined that Mycoplasma pneumoniae is present in the biological sample, and performing a next amplification cycle.

The kit of detecting Mycoplasma pneumoniae of the present disclosure can be used in various technologies based on a polymerase chain reaction. In a specific embodiment, the polymerase chain reaction can be qPCR. For example, the polymerase chain reaction can include 45 of the amplification cycles, and each of the amplification cycles comprises: a denaturation reaction performed at 95° C. for 5 seconds, and an annealing and extension reaction performed at 60° C. for 10 seconds. Preferably, an enzyme activation reaction can be performed prior to the amplification cycles, and the enzyme activation reaction can be performed at 95° C. for 30 seconds.

In order to further confirm that various embodiments of the present disclosure can be used to detect the presence of Mycoplasma pneumoniae and have the various advantages described above, the following tests are conducted.

### PREPARATION EXAMPLE 1

### Design of Primers and Probes

According to the data about Mycoplasma pneumoniae P1gene (accession number: LC753471.1) provided by the GenBank database, FIG. 1 shows the partial sequence of Mycoplasma pneumoniae P1 gene and the design positions of primers and probes.

In this embodiment, the forward primer is the nucleotide sequences shown as SEQ ID NOs: 1-4, which are complementary to the sequence after base 5421 of Mycoplasma pneumoniae P1 gene (as shown by the left arrow in FIG. 1); the reverse primer is the nucleotide sequences shown as SEQ ID NOs: 5-8, which are complementary to the sequence before base 5553 of Mycoplasma pneumoniae P1 gene (as shown by the right arrow in FIG. 1).

In this embodiment, the probe is the nucleotide sequence shown as SEQ ID NO: 9, which is complementary to the sequence between base 5471 and base 5494 of Mycoplasma pneumoniae P1 gene (as shown by the middle arrow in FIG. 1), and Cy5 is connected to its 5′ end.

### PREPARATION EXAMPLE 2

### Preparation of Plasmid of Mycoplasma pneumoniae P1 Gene

According to the LC753471.1 sequence shown in FIG. 1, it is selected and cloned into a vector to obtain a standard plasmid carrying Mycoplasma pneumoniae P1 gene (hereinafter referred to as the “P1 template”), and the plasmid containing only a vector is used as a control group (hereinafter referred to as the “control group”) for use in subsequent experiments. According to the present disclosure, any suitable vector can be used to construct the standard plasmid of Mycoplasma pneumoniae P1 gene, as long as it is replicable.

### PREPARATION EXAMPLE 3

### Screening of Primers and Probes

### A. Conservation of the Sequences of Primers and Probes

The nucleic acid sequences of the forward primer, reverse

primer and probe designed based on Mycoplasma pneumoniae P1 gene (i.e., SEQ ID NOs: 1-9) are analyzed on NCBI using Nucleotide Blast, and the primer sequences are analyzed by Primer Blast. As shown in Table 1, the analysis results confirm that the conservation of the sequences of SEQ ID NOs: 1-9 can reach more than 99.1. which has decent specificity and excludes the possibility that nucleic acid sequence fragments from other species can be identified.

TABLE 1

| | | | Number of | |
|---|---|---|---|---|
| Sequence | | | aligned | Conservation |
| number | Name | Sequence (5′ > 3′) | sequences | of sequence |
| SEQ ID | Forward | CGTGAAGTTCAAGTCTCCTG | 110 | 100% |
| NO: 1 | primer 1 | | | |
| SEQ ID | Forward | CAAGTCTCCTGACCAAATTGA | | 99.1% |
| NO: 2 | primer 2 | | | |
| SEQ ID | Forward | CACTGGTCCACAAAGCGT | | 100% |
| NO: 3 | primer 3 | | | |
| SEQ ID | Forward | TGGTCCACAAAGCGTGAA | | 100% |
| NO: 4 | primer 4 | | | |
| SEQ ID | Reverse | ACCAACATAGTTACCGGATCA | | 100% |
| NO: 5 | primer 1 | | | |
| SEQ ID | Reverse | GTACTGGTCATACACCAACATA | | 100% |
| NO: 6 | primer 2 | G | | |
| SEQ ID | Reverse | AACATAGTTACCGGATCAAACA | | 100% |
| NO: 7 | primer 3 | | | |
| SEQ ID | Reverse | AATAAACAGCGGTATGTACTGG | | 100% |
| NO: 8 | primer 4 | | | |
| SEQ ID | Probe | ACCGCTTGTTTACCCACCCAGTC | | 100% |
| NO: 9 | | A | | |

### B. Screening of the Sequences of Primers and Probes

In order to screen a combination of primers and probe with the best detection efficiency (Set 1 to Set 15 shown in Table 2) from the above forward primers, reverse primers and probe (shown in Table 1), a 106 copy number (cp) of the P1 template is prepared, and then it is diluted 10 times serially to prepare the following copy numbers: 104 cp, 102 cp, 101 cp; the forward primers, reverse primers and probe from Set 1 to Set 15 and a qPCR reaction reagent are added to various copy numbers of the PI template described above to produce a reaction solution; the reaction solution is put into a qPCR machine to maintain at 95° C. for 30 seconds for an enzyme activation phase, and then 45 cycles of the PCR phase are performed, wherein a denaturation at 95° C. for 5 seconds and an annealing and extension at 60° C. for 10 seconds are used in each cycle, with fluorescence detection at the end of each cycle. The overall PCR reaction time is 48 minutes. After the reaction, the threshold of cycle number (also known as quantification cycle, Cq) and amplification efficiency of Set 1 to Set 15 are compared. It can be seen from Table 2 that the groups with better amplification efficiency and earlier Cq value generation at low copy numbers (i.e., the reaction number is faster) include Set 1, Set 3, Set 5, Set 6, Set 7, Set 9 and Set 15. The above groups are then subjected to the second round of screening (as shown in Table 3). The human genome DNA (gDNA) is mixed with the P1 template and then mixed with the combination of primers and probe of each group in Table 3 to perform a qPCR reaction as described in Preparation Example 2. The P1 template without adding human gDNA is used as the gDNA-free control group. Each group is repeated twice to observe the situation under the interference of human gDNA. It can be seen from Table 3 that Set 5, Set 6, Set 7, and Set 9 are not interfered by human gDNA. Taking into account the time of Cq value generation, the range of A Cq, amplification efficiency, and any abnormal signal in the control group, the combination of primers and probe in Set 7 (SEQ ID NO: 2+SEQ ID NO: 8+SEQ ID NO: 9) is selected for subsequent testing due to the early generation of Cq value, the range of A Cq within ±1, the best amplification efficiency and no abnormal signal in the control group.

TABLE 2

| | Sequence number | | | | | | | | | | | | | | |
|---|---|---|---|---|---|---|---|---|---|---|---|---|---|---|---|
| Combination of | Set | Set | Set | Set | Set | Set | Set | Set | Set | Set | Set | Set | Set | Set | Set |
| primers and Probe | 1 | 2 | 3 | 4 | 5 | 6 | 7 | 8 | 9 | 10 | 11 | 12 | 13 | 14 | 15 |
| Former | No. 1 | | | No. 2 | | | | No. 3 | | | | No. 4 | | | |
| primer | | | | | | | | | | | | | | | |
| Reverse | No. 6 | No. 7 | No. 8 | No. 5 | No. 6 | No. 7 | No. 8 | No. 5 | No. 6 | No. 7 | No. 8 | No. 5 | No. 6 | No. 7 | No. 8 |
| primer | | | | | | | | | | | | | | | |
| Probe | No. 9 | | | | | | | | | | | | | | |
| 10⁶ cp/rxn | — | — | — | 21.21 | 20.65 | 21.05 | 19.51 | 21.78 | 21.31 | 21.55 | 19.92 | 21.55 | 21.04 | 21.4 | 19.67 |
| 10⁴ cp/rxn | — | — | — | 29.04 | 27.87 | 28.57 | 26.25 | 29.66 | 28.57 | 28.99 | 26.65 | 29.19 | 28.42 | 29.06 | 26.38 |
| 10² cp/rxn | — | — | — | ND | 35.58 | 35.46 | 33.19 | ND | 36.37 | 36.56 | 33.16 | 37.25 | 36.2 | 36.83 | 32.61 |
| | 34.61 | 35.26 | 33.01 | ND | 35.93 | 36.19 | 33.34 | ND | 35.91 | 36.49 | 33.75 | 37.47 | 36.42 | 36.89 | 33.08 |
| 10¹ cp/rxn | 37.00 | 38.38 | 35.27 | ND | 39.80 | 40.97 | 37.85 | NA | 40.13 | 41.12 | 34.95 | 41.68 | 42.59 | 42.29 | 38.65 |
| | 38.60 | 40.47 | 34.77 | ND | 40.56 | 40.31 | 38.11 | NA | 38.75 | 39.77 | 35.25 | NA | NA | 39.45 | 35.99 |
| Control | ND | ND | ND | ND | ND | ND | ND | ND | ND | ND | ND^{#} | ND | ND | ND | ND |
| group | | | | | | | | | | | | | | | |
| Efficiency | — | — | — | — | 84% | 90% | 95% | — | 86% | 85% | 98% | 80% | 83% | 81% | 101% |
| (%) | | | | | | | | | | | | | | | |

^{#}There is an abnormal increase on the signal, which may affect the detection specificity and may cause false positives;
ND: Non-Detect.

TABLE 3

| | Sequence number | | | | | | |
|---|---|---|---|---|---|---|---|
| Combination of | Set | Set | Set | Set | Set | Set | Set |
| primers and probe | 1 | 3 | 5 | 6 | 7 | 9 | 15 |
| Forward | No. 1 | | No. 2 | | | No. 3 | No. 4 |
| primer | | | | | | | |
| Reverse | No. 6 | No. 8 | No. 6 | No. 7 | No. 8 | No. 6 | No. 8 |
| primer | | | | | | | |
| Probe | No. 9 | | | | | | |
| Cq mean | 35.49 | 32.73 | 35.44 | 35.55 | 32.99 | 36.03 | 32.92 |
| (lpD 10² | | | | | | | |
| (control group)) | | | | | | | |
| n = 2 | | | | | | | |
| Cq mean | 36.74 | 34.17 | 34.50 | 34.73 | 33.53 | 35.68 | 34.29 |
| (lpD 10² + | | | | | | | |
| gDNA 200 ng) | | | | | | | |
| n = 2 | | | | | | | |
| Δ Cq (gDNA | 1.25 | 1.44 | −0.94 | −0.82 | 0.54 | −0.35 | 1.37 |
| 200 ng- | | | | | | | |
| control group) | | | | | | | |

### EXAMPLE 1

### Sensitivity Analysis of the Kit of Detecting Mycoplasma pneumoniae of the Present Disclosure

The different copy numbers of P1 template are used in this example to test the sensitivity of the kit of detecting Mycoplasma pneumoniae of the present disclosure including the specific combination of primers and probe (SEQ ID No: 2+SEQ ID No: 8+SEQ ID No: 9). First, a 106 cp of the P1 template is prepared, and then it is diluted 10 times serially to prepare the following copy numbers: 105 cp, 104 cp, 103 cp, 102 cp, 101 cp, followed by the qPCR reaction as described in Preparation Example 2.

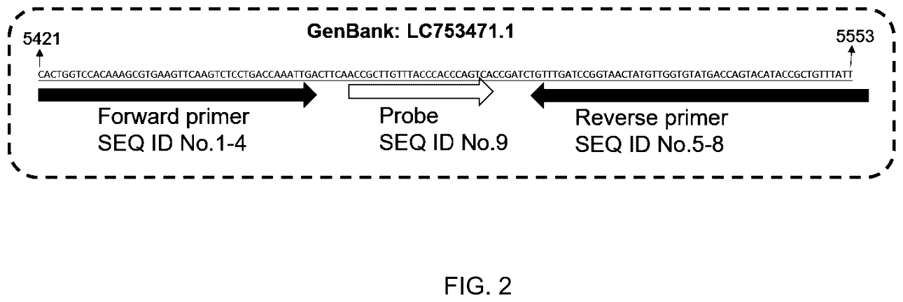

Refer to FIGS. 2A and 2B, which are an amplification curve graph and a standard curve graph obtained after the qPCR reaction. As shown in FIG. 2A, there is no amplification product in the control group (i.e., the lower horizontal line in FIG. 2A), and thus there is no fluorescent reaction. On the contrary, there is a positive trend in the fluorescence reaction results of 101 cp to 106 cp.

Continue to refer to FIG. 2B, which is a standard curve graph using the amplified result values in FIG. 2A. The horizontal axis is the logarithmic value of the copy numbers, which is the Log starting quantity, and the vertical axis is the Cq value. Generally, amplification efficiency ranges from 90% to 110%. As shown in FIG. 2B, based on the aforementioned result values of 101 cp to 106 cp, a straight line with a slope of 3.179 can be drawn, with a correlation coefficient of 0.996 and an amplification efficiency of 106.3%. Therefore, the sensitivity of the kit of detecting Mycoplasma pneumoniae including the specific combination of primers and probe (SEQ ID No: 2+SEQ ID No: 8+SEQ ID No: 9) of the present disclosure is within the range of the amplification efficiency.

### EXAMPLE 2

### Specificity Analysis of the Kit of Detecting Mycoplasma pneumoniae of the Present Disclosure

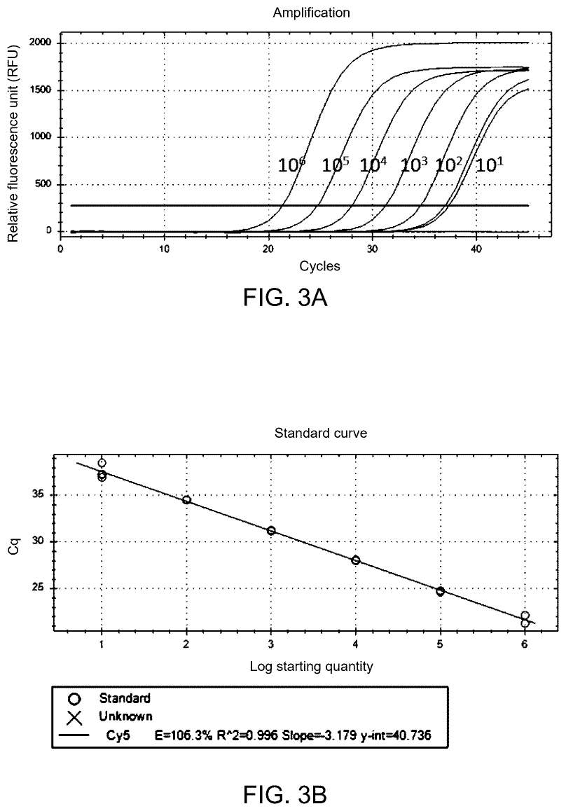

The qPCR reaction described in Preparation Example 2 is used in this example to test the specificity of the kit of detecting Mycoplasma pneumoniae including the specific combination of primers and probe (SEQ ID No: 2+SEQ ID No: 8+SEQ ID No: 9) of the present disclosure. The following copy numbers of the P1 template are prepared: 20 cp, 100 cp, and 10000 cp, followed by the qPCR reaction. The amplification products obtained are analyzed by gel electrophoresis. The results are shown in FIG. 3.

It can be seen from FIG. 3 that there is no amplification product in the result of 0 cp. The size of the amplification products of 20 cp, 100 cp and 10000 cp is about 109 bp, and there is no non-specific product. Therefore, the kit of detecting Mycoplasma pneumoniae including the specific combination of primers and probe (SEQ ID No: 2+SEQ ID No: 8+SEQ ID No: 9) of the present disclosure has a high specificity, and there is no non-specific amplification.

### EXAMPLE 3

### Interference Test of the Kit of Detecting Mycoplasma pneumoniae of the Present Disclosure

In order to test whether human gDNA interfere with the detection of Mycoplasma pneumoniae and thus affect the detection results, 200 ng of human gDNA is added to a biological sample containing Mycoplasma pneumoniae, and DELBio G1-Automatic Nucleic Acid Testing is used to sequentially perform the extraction and purification of Mycoplasma pneumoniae genes, the qPCR amplification of Mycoplasma pneumoniae genes, the fluorescence detection and result analysis. Two duplicate tests are performed. As shown in Table 4, the results show that compared with the control group, 1000 copy/rxn of the biological sample containing Mycoplasma pneumoniae, 100 copy/rxn of the biological sample containing Mycoplasma pneumoniae, and 20 copy/rxn of the biological sample containing Mycoplasma pneumoniae all can be detected by the kit of detecting Mycoplasma pneumoniae of the present disclosure. Moreover, there is no difference in Cq values between the two groups of 100 copy/rxn of the biological sample containing Mycoplasma pneumoniae and 100 copy/rxn of the biological sample containing Mycoplasma pneumoniae added with 200 ng human gDNA, indicating that the use of the kit of detecting Mycoplasma pneumoniae including the specific combination of primers and probe (SEQ ID No: 2+SEQ ID No: 8+SEQ ID No: 9) of the present disclosure can detect biological samples containing different copy numbers of Mycoplasma pneumoniae and biological samples containing Mycoplasma pneumoniae with the interference of 200 ng gDNA, and the detection sensitivity reach to 100 copy/rxn under the interference of 200 ng gDNA.

TABLE 4

| | Cy5 |
|---|---|
| Copy/rxn | Cq |
| 10000 | 26.25 |
| | 26.29 |
| 100 | 32.86 |
| | 33.08 |
| 20 | 35.73 |
| | 35.46 |
| Control group | NaN |
| | NaN |
| 100 copy + gDNA 200 ng | 33.05 |
| | 33.00 |

### EXAMPLE 4

### Efficiency Test of the Kit of Detecting Mycoplasma pneumoniae of the Present Disclosure in the Form of a Freeze-Dried Reagent

In order to test whether the freeze-drying of the reagents of the kit of detecting Mycoplasma pneumoniae containing the specific combination of primers and probe (SEQ ID No: 2+SEQ ID No: 8+SEQ ID No: 9) affect the detection efficiency of the kit. 100 cp and 20 cp of the PI template are prepared respectively, and the kit of detecting Mycoplasma pneumoniae in the form of a liquid reagent and the kit of detecting Mycoplasma pneumoniae in the form of a freeze-dried reagent after reconstitution are used to perform the qPCR reaction as described in Preparation Example 2. Two duplicate tests are performed, and the Cq value and ACq range are compared. The results are shown in Table 5.

It can be seen from Table 5 that when the kit of detecting Mycoplasma pneumoniae in the form of a liquid reagent or in the form of a freeze-dried reagent is used to detect the 100 cp and 20 cp of the P1 template, there is no difference in the Cq value, and the range of ACq is within #1, showing that the efficiency of the kit of detecting Mycoplasma pneumoniae of the present disclosure after freeze-drying is not affected.

TABLE 5

| | | | ΔCq (Cq value of the |
|---|---|---|---|
| | Cq value | Cq value | freeze-dried reagent − |
| | (the liquid | (the freeze- | Cq value of the |
| Cy5 | reagent) | dried reagent) | liquid reagent) |
| 100 cp | 32.86 | 33.13 | 0.02 |
| | 33.08 | 32.85 | |
| 20 cp | 35.73 | 36.15 | 0.38 |
| | 35.46 | 35.79 | |
| Control | NaN | NaN | — |
| group | NaN | NaN | |

### EXAMPLE 5

### Limit of Detection (LoD) of the Kit of Detecting Mycoplasma pneumoniae of the Present Disclosure

In order to determine the LoD of the kit of detecting Mycoplasma pneumoniae containing the specific combination of primers and probe (SEQ ID No: 2 +SEQ ID No: 8 +SEQ ID No: 9) of the present disclosure, 20 copy/rxn, 10 copy/rxn and 5 copy/rxn of the P1 template are prepared respectively, and 105 copy/rxn of human genes are added as an internal control group. The kit of detecting Mycoplasma pneumoniae of the present disclosure is used to perform the qPCR reaction as described in Preparation Example 2. 20 copy/rxn of the P1 template is subjected to 12 duplicate tests, 10 copy/rxn and 5 copy/rxn of the P1 template are subjected to 6 duplicate tests, and fluorescence detection (the fluorescent probe of the internal control group is connected with HEX reporter) and result analysis are finally performed, as shown in Table 6.

It can be seen from Table 6 that the 12 duplicate tests of 20 copy/rxn of the P1 template with 105 copy/rxn of human genes, the 6 duplicate tests of 10 copy/rxn of the P1 template with 105 copy/rxn of human genes and the 6 duplicate tests of 5 copy/rxn of the P1 template with 105 copy/rxn of human genes all can be detected by the kit of detecting Mycoplasma pneumoniae of the present disclosure, showing that in the presence of high concentration of human genes, the LoD of the kit of detecting Mycoplasma pneumoniae containing the specific combination of primers and probe (SEQ ID No: 2+SEQ ID No: 8+SEQ ID No: 9) of the present disclosure can be as low as 5 copy/rxn.

TABLE 6

| Copy number of the | 20 copy/rxn | 10 copy/rxn | 5 copy/rxn | Control | LOD 95% |
|---|---|---|---|---|---|
| P1 template | | | | group | |
| Human genes | 10⁵ copy/rxn | | | | (Probit |
| (RNase P gene; | | | | | analysis) |
| served as the | | | | | |
| internal control | | | | | |
| group) | | | | | |
| the kit of detecting | 12/12 | 6/6 | 6/6 | 0/6 | 5 copy/rxn |
| Mycoplasma | | | | | |
| pneumoniae of the | | | | | |
| present disclosure | | | | | |
| (Cy5) | | | | | |
| Internal control | 12/12 | 6/6 | 6/6 | 0/6 | |
| group (HEX) | | | | | |

Given the above, the advantages of the present disclosure are that:
(1) High specificity: the sequence coverage rate of primers and probes is maintained at more than 99%, gene sequence fragments of other species other than Mycoplasma pneumoniae is not identified, and thus the kit has a high degree of specificity;
(2) High sensitivity: the limit of detection (LoD) of this primer-probe combination is 5 copy/rxn nucleic acid samples; and when there is interference from human genome DNA, this primer-probe combination can still detect 100 copy/rxn nucleic acid samples;
(3) Fast reaction time: compared with other detection kits in the industry, the detection kit of the present disclosure has a reaction time of less than 1 hour, and the results can be obtained quickly; and
(4) Cost reduction: after the primer-probe combination is freeze-dried, the reaction efficiency is not affected, and the cold chain cost during transportation can be reduced.

Although the present disclosure has been disclosed in preferred embodiments, those embodiments are not intended to limit the present disclosure. Any person skilled in the art can make various changes and modifications without departing from the spirit and scope of the present disclosure. Therefore, the protection scope of the present disclosure shall be determined by the appended claims.

1. A kit of detecting Mycoplasma pneumoniae, comprising a combination of primers and probe consisting of a forward primer, a reverse primer and a probe, wherein the forward primer is selected from the group consisting of SEQ ID NO: 2-3 and complementary sequences thereof, the reverse primer is selected from the group consisting of SEQ ID NO: 6-8 and complementary sequences thereof, and the probe is SEQ ID NO: 9 or a complementary sequence thereof.

2. The kit of claim 1, wherein the forward primer is SEQ ID NO: 2, and the reverse primer is SEQ ID NO: 8.

3. The kit of claim 1, wherein the 5′ end of the probe is connected to a reporter, and the 3′ end of the probe is connected to a quencher.

4. The kit of claim 3, wherein the reporter is Cy5.

5. The kit of claim 1, wherein the combination of primers and probe is in a form of a freeze-dried reagent.

6. A method for detecting Mycoplasma pneumoniae, comprising steps of:
S110: providing a biological sample;
S120: providing the kit of detecting Mycoplasma pneumoniae as claimed in claim 1;
S130: mixing the biological sample and the kit of detecting Mycoplasma pneumoniae to obtain a reaction solution;
S140: amplifying nucleic acids in the biological sample by performing a polymerase chain reaction using the reaction solution to obtain an amplification product; and
S150: detecting a fluorescent signal after each amplification cycle, analyzing the amplification product based on the fluorescent signal to determine whether Mycoplasma pneumoniae is present in the biological sample, wherein when the fluorescent signal is detected, it is determined that Mycoplasma pneumoniae is present in the biological sample; when the fluorescent signal is not detected, it is not yet determined that Mycoplasma pneumoniae is present in the biological sample, and performing a next amplification cycle.

7. The method of claim 6, wherein the polymerase chain reaction is real-time quantitative polymerase chain reaction.

8. The method of claim 6, wherein the polymerase chain reaction includes 40 to 50 of the amplification cycles.

9. The method of claim 8, wherein each of the amplification cycles comprises: a denaturation reaction, performed at 93 to 97° C. for 4 to 6 seconds;
and an annealing and extension reaction, performed at 58 to 62° C. for 8 to 12 seconds.

10. The method of claim 8, wherein an enzyme activation reaction is performed prior to each of the amplification cycles, and the enzyme activation reaction is performed at 93 to 97° C. for 25 to 35 seconds.


SEQUENCE LISTING (9 sequences)

SEQ ID NO: 1 - DNA, 20 bp, synthetic construct
  source 1..20: mol_type = other DNA; note = forward primer
cgtgaagttcaagtctcctg

SEQ ID NO: 2 - DNA, 21 bp, synthetic construct
  source 1..21: mol_type = other DNA; note = forward primer
caagtctcctgaccaaattga

SEQ ID NO: 3 - DNA, 18 bp, synthetic construct
  source 1..18: mol_type = other DNA; note = forward primer
cactggtccacaaagcgt

SEQ ID NO: 4 - DNA, 18 bp, synthetic construct
  source 1..18: mol_type = other DNA; note = forward primer
tggtccacaaagcgtgaa

SEQ ID NO: 5 - DNA, 21 bp, synthetic construct
  source 1..21: mol_type = other DNA; note = reverse primer
accaacatagttaccggatca

SEQ ID NO: 6 - DNA, 23 bp, synthetic construct
  source 1..23: mol_type = other DNA; note = reverse primer
gtactggtcatacaccaacatag

SEQ ID NO: 7 - DNA, 22 bp, synthetic construct
  source 1..22: mol_type = other DNA; note = reverse primer
aacatagttaccggatcaaaca

SEQ ID NO: 8 - DNA, 22 bp, synthetic construct
  source 1..22: mol_type = other DNA; note = reverse primer
aataaacagcggtatgtactgg

SEQ ID NO: 9 - DNA, 24 bp, synthetic construct
  source 1..24: mol_type = other DNA; note = probe
accgcttgtttacccacccagtca